(19) United States
(12) Patent Application Publication
(10) Pub. No.: US 2004/0202708 A1
(43) Pub. Date: Oct. 14, 2004

(54) Transdermal drug delivery device with translucent inorganic barrier layer

(75) Inventors: Mark A. Roehrig, La Mesa, CA (US); Christopher S. Lyons, St. Paul, MN (US); James B. Barnhart, Afton, MN (US)

Correspondence Address: 3M INNOVATIVE PROPERTIES COMPANY, PO BOX 33427, ST. PAUL, MN, 55133-3427, US

(73) Assignee: 3M Innovative Properties Company

(21) Appl. No.: 10/823,328

(22) Filed: Apr. 13, 2004

Related U.S. Application Data
(60) Provisional application No. 60/462,718, filed on Apr. 14, 2003.

(51) Int. Cl. A61K 9/70

(52) U.S. Cl. 424/449

(57) ABSTRACT

The present invention comprises a transdermal drug delivery device for delivering a pharmaceutically active agent comprising a reservoir comprising a releasably stored dosage of the pharmaceutically active agent and a substantially continuous, translucent inorganic barrier layer adjacent to at least a portion of the reservoir. The present invention also comprises a method of drug delivery to a mammal using such devices.

This application claims priority to U.S. Provisional Patent Application No. 60/462718, filed Apr. 14, 2003.

### FIELD OF THE INVENTION

The present invention relates to a transdermal drug delivery device with a translucent inorganic barrier layer.

### BACKGROUND OF THE INVENTION

Transdermal drug delivery is a well known method for administering pharmaceuticals. Transdermal drug delivery devices typically consist of a reservoir containing a drug. An example of such a reservoir is an adhesive matrix that has a drug dispersed or dissolved throughout the matrix. The adhesive matrix is placed in contact with a skin surface when in use and the drug passes from the device into and through the skin. Such devices typically have a backing material that protects the portion of the reservoir that is not in contact with the skin.

It is often desired that a backing material limit moisture and oxygen transmission through the device, as well as limiting diffusion of components of the reservoir formulation into or through the backing material. It is also often desired that a backing material be flexible and translucent. Typical flexible and translucent materials used in transdermal devices, such as polyethylene film, have limitations in their ability to limit moisture and oxygen transmission. Typical barriers used in transdermal devices, such as laminates including aluminum foil, have limitations in their flexibility and are not translucent.

### SUMMARY OF THE INVENTION

It is one object of the present invention to provide a transdermal drug delivery device having a translucent barrier to moisture and oxygen transmission.

In one aspect, the present invention comprises a transdermal drug delivery device for delivering a pharmaceutically active agent comprising a reservoir comprising a releasably stored dosage of the pharmaceutically active agent and a substantially continuous, translucent inorganic barrier layer adjacent to at least a portion of the reservoir.

In another aspect, the present invention comprises a method of drug delivery to a mammal comprising providing a reservoir comprising a pharmaceutically active agent, providing a substantially continuous, translucent inorganic barrier layer adjacent to at least a portion of one surface of the reservoir, placing the surface of the reservoir opposed to the surface adjacent to the inorganic barrier layer in a delivering relationship to the skin surface of the mammal, and allowing the reservoir to remain in a delivering relationship to the skin for a period of time sufficient to provide a therapeutic effect.

The above summary of the present invention is not intended to describe each disclosed embodiment or every implementation of the present invention. The Figures and the detailed description that follow more particularly exemplify illustrative embodiments.

### BRIEF DESCRIPTION OF THE DRAWINGS

Preferred embodiments of the invention will now be described in greater detail below with reference to the attached drawings, wherein:

FIG. 1 shows a schematic cross-section of an embodiment of the present invention where the inorganic barrier layer directly adjoins the reservoir.

FIG. 2 shows a schematic cross-section of an embodiment of the present invention where the inorganic barrier layer directly adjoins a backing.

FIG. 3 shows a schematic cross-section of an embodiment of the present invention having optional polymer layers surrounding the inorganic barrier layer.

FIG. 4 shows a schematic cross-section of an embodiment of the present invention having a matrix reservoir.

### DETAILED DESCRIPTION OF THE INVENTION

In one embodiment, the present invention comprises a transdermal drug delivery device for delivering a pharmaceutically active agent comprising a reservoir comprising a releasably stored dosage of the pharmaceutically active agent and a substantially continuous, translucent inorganic barrier layer adjacent to at least a portion of the reservoir.

The reservoir serves the basic function of containing a pharmaceutically active agent. Transdermal drug delivery device comprising reservoirs are well-known and include: devices containing gelled or liquid reservoirs, such as in U.S. Pat. No. 4,834,979 (Gale), so-called “reservoir” patches; devices containing matrix reservoirs attached to the skin by an adjacent adhesive layer, such as in U.S. Pat. No. 6,004,578 (Lee, et al.), so-called “matrix” patches; and devices containing pressure-sensitive adhesive reservoirs, such as in U.S. Pat. No. 6,365,178 (Venkateshwaran et al.), so-called “drug-in-adhesive” patches, the disclosures of which are incorporated herein by reference. In each instance, it is preferred that the reservoir of the patch be protected in some manner from the outside environment.

Exemplary pharmaceutically active agents (also referred to here as drugs) that can be included in the reservoir include any substance capable of local or systemic effect when administered to the skin, such as clonidine, estradiol, nicotine, nitroglycerine, scopolamine, and fentanyl, all of which are commercially available in the form of transdermal devices. Others include antiinflammatory drugs, both steroidal (e.g., hydrocortisone, prednisolone, triamcinolone) and nonsteroidal (e.g., naproxen, piroxicam); bacteriostatic agents (e.g., chlorhexidine, hexylresorcinol); antibacterials (e.g., penicillins such as penicillin V, cephalosporins such as cephalexin, erythromycin, tetracycline, gentamycin, sulfathiazole, nitrofurantoin, and quinolones such as norfloxacin, flumequine, and ibafloxacin); antiprotazoals (e.g., metronidazole); antifungals (e.g., nystatin); coronary vasodilators; calcium channel blockers (e.g., nifedipine, diltiazem); bronchodilators (e.g., theophylline, pirbuterol, salmeterol, isoproterenol); enzyme inhibitors such as collagenase inhibitors, protease inhibitors, elastase inhibitors, lipoxygenase inhibitors (e.g., A64077), and angiotensin converting enzyme inhibitors (e.g., captopril, lisinopril); other antihypertensives (e.g., propranolol); leukotriene antagonists (e.g., ICI204,219); anti-ulceratives such as H2 antagonists; steroidal hormones (e.g., progesterone, testosterone, estradiol); antivirals and/or immunomodulators (e.g., 1-isobutyl-1H-imidazo[4,5-c]quinolin-4-amine, 1-(2-hydroxy-2-methylpropyl)-1H-imidazo[4,5-c]quinolin-4-amine, and acyclovir); local anesthetics (e.g., benzocaine, propofol); cardiotonics (e.g., digitalis, digoxin); antitussives (e.g., codeine, dextromethorphan); antihistamines (e.g., diphenhydramine, chlorpheniramine, terfenadine); narcotic analgesics (e.g., morphine, buprenorphine); peptide hormones (e.g., human or animal growth hormones, LHRH); cardioactive products such as atriopeptides; proteinaceous products (e.g., insulin); enzymes (e.g., anti-plaque enzymes, lysozyme, dextranase); antinauseants; anticonvulsants (e.g., carbamazine); immunosuppressives (e.g., cyclosporine); psychotherapeutics (e.g., diazepam); sedatives (e.g., phenobarbital); anticoagulants (e.g., heparin); analgesics (e.g., acetaminophen); antimigraine agents (e.g., ergotamine, melatonin, sumatripan); antiarrhythmic agents (e.g., flecainide); antiemetics (e.g., metaclopromide, ondansetron); anticancer agents (e.g., methotrexate); neurologic agents such as anxiolytic drugs; hemostatics; anti-obesity agents; and the like, as well as pharmaceutically acceptable salts and esters thereof. The amount of drug that constitutes a therapeutically effective amount can be readily determined by those skilled in the art with due consideration of the particular drug, the particular carrier, and the desired therapeutic effect.

The reservoir may optionally contain other additives or excipients in addition to the drug and the carrier matrix. Such additives include pharmaceutically acceptable materials that may be used as skin penetration enhancers (i.e., substances that increase the permeation rate a drug across or into the skin) or solubilizers (i.e., substances that effectively solubilize a drug) in transdermal drug delivery systems. Exemplary materials include C₈-C₂₀ fatty acids such as isostearic acid, octanoic acid, and oleic acid; C₈-C₂₀ fatty alcohols such as oleyl alcohol and lauryl alcohol; lower alkyl esters of C₈-C₂₀ fatty acids such as ethyl oleate, isopropyl myristate, butyl stearate, and methyl laurate; di(lower) alkyl esters of C₆-C₈ diacids such as diisopropyl adipate; monoglycerides of C₈-C₂₀ fatty acids such as glyceryl monolaurate; tetraglycol (tetrahydrofurfuryl alcohol polyethylene glycol ether); tetraethylene glycol (ethanol,2,2′-(oxybis(ethylenoxy))diglycol); C₆-C₂₀ alkyl pyrrolidone carboxylates; polyethylene glycol; propylene glycol; 2-(2-ethoxyethoxy)ethanol; diethylene glycol monomethyl ether; N,N-dimethyldodecylamine-N-oxide and combinations of the foregoing. Alkylaryl ethers of polyethylene oxide, polyethylene oxide monomethyl ethers, polyethylene oxide dimethyl ethers, glycerol, and N-methyl pyrrolidone are also suitable. The terpenes are another useful class of pharmaceutical excipients, including pinene, d-limonene, carene, terpineol, terpinen-4-ol, carveol, carvone, pulegone, piperitone, menthone, menthol, neomenthol, thymol, camphor, borneol, citral, ionone, and cineole, alone or in any combination.

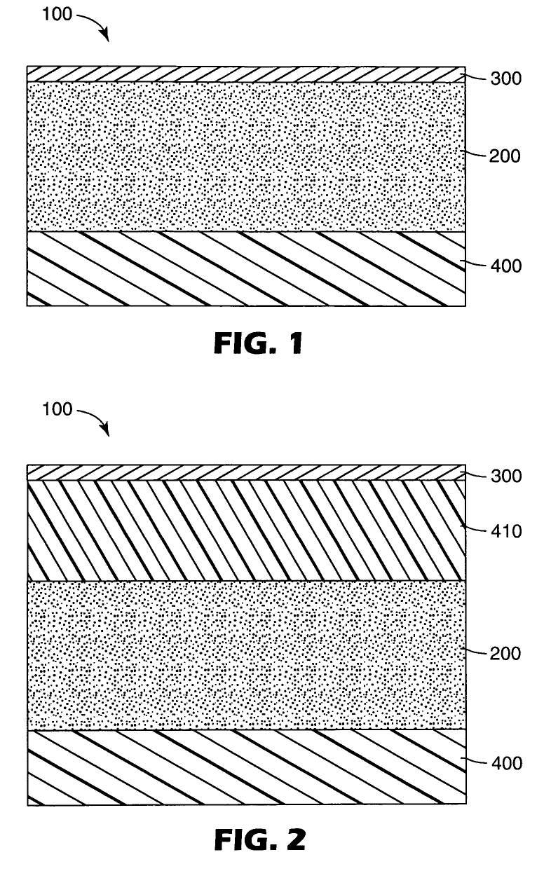

In one aspect, the invention comprises a substantially continuous, translucent inorganic barrier layer adjacent to at least a portion of the reservoir. One example of a substantially continuous, translucent inorganic barrier layer adjacent to at least a portion of the reservoir is shown schematically in FIG. 1. In the embodiment shown in FIG. 1, the device 100 has a pressure-sensitive adhesive reservoir 200 comprising a therapeutically active agent. The substantially continuous, translucent inorganic barrier layer 300 directly adjoins the pressure-sensitive adhesive reservoir 200. As shown, the device further has a release liner 400. Prior to use by a patient the release liner 400 protects the surface of the pressure-sensitive adhesive reservoir 200 opposed to the inorganic barrier layer 300, which would otherwise be exposed. In use, the release liner 400 is removed and the pressure-sensitive adhesive reservoir 200 is adhered to a skin surface.

The inorganic barrier layer is adjacent to at least a portion of the reservoir. As shown in FIG. 1, the inorganic barrier layer 300 directly adjoins the reservoir 200 and covers one entire surface of the reservoir 200. By adjacent, however, it should be understood that the inorganic barrier layer need not be in direct contact with the reservoir, but may be separated from the reservoir by other layers, for example tie layers, backing layers, flat electrodes or other polymeric film layers.

The inorganic barrier layer is substantially continuous and translucent. By substantially continuous it should be understood that the inorganic barrier layer is intended to cover an area with a continuous coating, that is, it is not intended to be in the form of discrete, non-contiguous particles. It should be understood, however, that preparation of a substantially continuous layer may still leave occasional microscopic defects in the layer, which may allow a through-path for transport of vapors or fluids that results in a transport rate in the microscopic areas that is higher than the average transport rate across the inorganic barrier layer. By translucent it should be understood that the inorganic barrier layer is non-opaque, or alternatively that the inorganic barrier layer is able to transmit some portion of visible light across the layer. Typically, the average visible transmittance is at least 20%. Average visible transmittance is defined as the average of the measured visible (i.e., 400 nm to 700 nm) transmittance spectrum at 0 degree (normal) angle of incidence. In a preferred embodiment, the aggregate of other layers or other structures within the transdermal delivery device are also translucent.

The inorganic barrier layer typically consists of metal oxides, metal nitrides, metal oxy-nitrides, and metal alloys of oxides, nitrides and oxy-nitrides. In one aspect the inorganic barrier layer comprises a metal oxide. Preferred metal oxides include indium tinoxide, aluminum oxide, silicon oxide, aluminum-silicon-oxide, aluminum-silicon-nitride, and aluminum-silicon-oxy-nitride. The inorganic barrier layers may be prepared by a variety of methods, such as those described in U.S. Pat. No. 5,725,909 (Shaw et al.) and U.S. Pat. No. 5,440,446 (Shaw et al.), the disclosures of which are incorporated by reference. Inorganic barrier layers can typically be prepared by reactive evaporation, reactive sputtering, chemical vapor deposition and plasma enhanced chemical vapor deposition. Preferred methods include vacuum preparations such as reactive sputtering and plasma enhanced chemical vapor deposition

The inorganic barrier layers are typically thin layers, with a preferred thickness of less than 200 nm, more preferably less than 150 nm, and most preferably less than 100 nm. The inorganic barrier layers are typically thicker than about 10 nm, with a preferred thickness of more than about 30 nm.

In one aspect, devices of the present invention have a single inorganic barrier layer. In another aspect, devices of the present invention may have a plurality of inorganic barrier layers. The materials and methods used to prepare the plurality of inorganic barrier layers may be independently selected. The thickness of each inorganic barrier layer need not be the same. In one aspect, the plurality of inorganic barrier layers are separated from each other by intervening layers comprising a polymer.

In one aspect, devices of the present invention may optionally comprise at least one additional layer comprising a polymer. Acrylate and methacrylate polymers are particularly preferred as the polymer of the at least one additional layer. The group of acrylates and methacrylates will also be referred to as “(meth)acrylates”.

In one aspect, the polymer layer can be applied using conventional coating methods such as roll coating (e.g., gravure roll coating) or spray coating (e.g., electrostatic spray coating), then crosslinked using, for example, UV radiation. The polymer layer is preferably formed by flash evaporation, vapor deposition and crosslinking of a monomer as described in U.S. Pat. Nos. 4,842,893 (Yializis et al.); 4,954,371 (Yializis); 5,032,461 (Shaw et al.); 5,440,446 (Shaw et al.); 5,725,909 (Shaw et al.); 6,231,939 (Shaw et al); 6,045,864 (Lyons et al.); and 6,224,948 (Affinito), the disclosures of which are incorporated by reference. Volatilizable (meth)acrylate monomers are preferred for use in such a process, with volatilizable acrylate monomers being especially preferred. Preferred (meth)acrylates have a number average molecular weight in the range of about 150 to about 600, more preferably about 200 to about 400. Other preferred (meth)acrylates have a value of the ratio of the molecular weight to the number of acrylate functional groups per molecule in the range of about 150 to about 600 g/mole/(meth)acrylate group, more preferably about 200 to about 400 g/mole/(meth)acrylate group. Fluorinated (meth)acrylates can be used at higher molecular weight ranges or ratios, e.g., about 400 to about 3000 molecular weight or about 400 to about 3000 g/mole/(meth)acrylate group. Coating efficiency can be improved by cooling the coating substrate. Preferred monomers include multifunctional (meth)acrylates, used alone or in combination with other multifunctional or monofunctional (meth)acrylates. Examples of suitable monomers include, but are not limited to, hexanediol diacrylate, ethoxyethyl acrylate, phenoxyethyl acrylate, cyanoethyl (mono)acrylate, isobornyl acrylate, isobornyl methacrylate, octadecyl acrylate, isodecyl acrylate, lauryl acrylate, beta-carboxyethyl acrylate, tetrahydrofurfuryl acrylate, dinitrile acrylate, pentafluorophenyl acrylate, nitrophenyl acrylate, 2-phenoxyethyl acrylate, 2-phenoxyethyl methacrylate, 2,2,2-trifluoromethyl (meth)acrylate, diethylene glycol diacrylate, triethylene glycol diacrylate, triethylene glycol dimethacrylate, tripropylene glycol diacrylate, tetraethylene glycol diacrylate, neopentyl glycol diacrylate, propoxylated neopentyl glycol diacrylate, polyethylene glycol diacrylate, tetraethylene glycol diacrylate, bisphenol A epoxy diacrylate, 1,6-hexanediol dimethacrylate, trimethylol propane triacrylate, ethoxylated trimethylol propane triacrylate, propylated trimethylol propane triacrylate, tris(2-hydroxyethyl)-isocyanurate triacrylate, pentaerythritol triacrylate, phenylthioethyl acrylate, naphthloxyethyl acrylate, IRR-214 cyclic diacrylate from UCB Chemicals, epoxy acrylate RDX80095 from Rad-Cure Corporation, and mixtures thereof. A variety of other curable materials can be included in the crosslinked polymeric layer, e.g., vinyl ethers, vinyl naphthalene, acrylonitrile, and mixtures thereof.

In one aspect, a polymer layer may provide a highly smooth surface on which one or more inorganic barrier layers may be deposited. In another aspect, a polymer layer may protect an underlying inorganic barrier layer from abrasion or rough handling.

The polymer layers are typically thin layers, with a preferred thickness of about 1000 nm or less. The polymer layers are typically thicker than about 10 nm, with a preferred thickness of about 100 nm or more.

Transdermal drug delivery devices of the invention can be made in the form of an article such as a tape, a patch, a sheet, a dressing or any other form known to those skilled in the art. Generally, the device will be in the form of a patch of a size suitable to deliver a selected amount of drug through the skin.

Generally, the device will have a surface area greater than about 1 cm², and more typically greater than about 5 cm². Generally, the device will have a surface area of less than about 100 cm², preferably less than about 40 cm².

In one aspect, devices of the present invention comprise a backing film substrate (or backing). Typical examples of flexible backings employed as conventional tape backings which may be useful for the present invention include those made from polymer films such as polypropylene; polyethylene, particularly low density polyethylene, linear low density polyethylene, metallocene polyethylenes, and high density polyethylene; polyvinyl chloride; polyester (e.g., polyethylene terephthalate); ethylene-vinyl acetate copolymer; polyurethane; cellulose acetate; and ethyl cellulose. Fabrics and non-wovens are also suitable. Coextruded multilayer polymeric films are also suitable, such as those described in U.S. Pat. No. 5,783,269 (Heilmann et al.), the disclosure of which is incorporated herein by reference.

In one aspect, the inorganic barrier layer may be deposited directly onto one surface of the backing. In another aspect, one or more intervening layers is present between the inorganic barrier layer and the adjacent surface of the backing. Preferably, the one or more intervening layers comprises a polymer layer, such as a crosslinked acrylate layer. It is preferred that backing substrates coated with an inorganic barrier layer and an optional polymer layer are flexible, more preferably having substantially the same flexibility as the uncoated backing substrate.

The backing thickness is preferably more than 10 μm, more preferably more than 20 μm, and most preferably more than 40 μm. The backing thickness is preferably less than 150 μm, more preferably less than 125 μm, and most preferably less than 100 μm.

In one aspect, devices of the present invention comprise a release liner that covers and protects the skin-contacting surface prior to use by a patient. Suitable release liners include conventional release liners comprising a known sheet material such as a polyester web, a polyethylene web, a polypropylene web, or a polyethylene-coated paper coated with a suitable fluoropolymer or silicone based coating. Devices of the present invention may be packaged individually in a foil-lined pouch for storage. Devices of the present invention may alternatively be provided in a rolled or stacked form suitable for use with a dispensing apparatus.

In the embodiment shown in FIG. 2, the device 100 has a pressure-sensitive adhesive reservoir 200 comprising a therapeutically active agent. The substantially continuous, translucent inorganic barrier layer 300 directly adjoins a backing 410, which in turn adjoins the pressure-sensitive adhesive reservoir 200. The device further has a release liner 400.

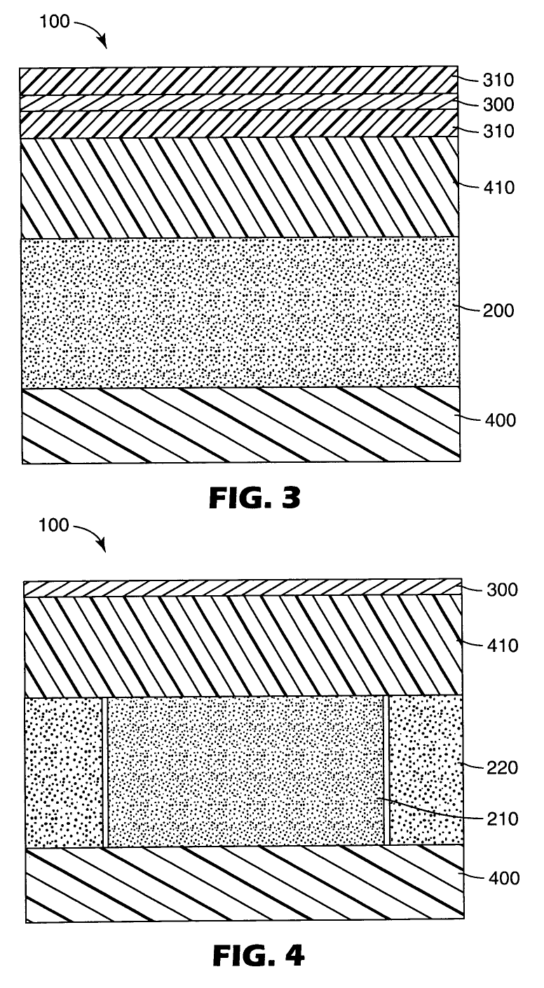

In the embodiment shown in FIG. 3, the device 100 is similar to that shown in FIG. 2, except that additional polymer layers 310 are present surrounding the inorganic barrier layer 300.

In the embodiment shown in FIG. 4, the device 100 is similar to that shown in FIG. 1, except that it comprises a non-pressure-sensitive adhesive matrix reservoir 210 that is surrounded by an outer edge of pressure-sensitive adhesive 220 that serves to adhere the device to a skin surface. As shown, there is a slight air gap between the matrix reservoir 210 and the outer edge of pressure-sensitive adhesive 220. Alternatively, the matrix reservoir 210 and the outer edge of pressure-sensitive adhesive 220 may be directly in contact with each other.

In another aspect, the present invention comprises a method of drug delivery to a mammal comprising providing a reservoir comprising a pharmaceutically active agent, providing a substantially continuous, translucent inorganic barrier layer adjacent to at least a portion of one surface of the reservoir, placing the surface of the reservoir opposed to the surface adjacent to the inorganic barrier layer in a delivering relationship to the skin surface of the mammal, and allowing the reservoir to remain in a delivering relationship to the skin for a period of time sufficient to provide a therapeutic effect. The reservoir may be placed in direct contact with the skin surface, such as where the reservoir comprises a pressure-sensitive adhesive. Alternatively, the reservoir may be separated from the skin surface by a membrane or other layer that moderates or controls the delivery of the drug to the skin surface. The length of time that the reservoir remains in a delivering relationship is typically an extended time, preferably from about 12 hours to about 14 days. The length of time that the reservoir remains in a delivering relationship is preferably about 1 day (i.e., daily dosing), about 3 to 4 days (bi-weekly dosing), or about 7 days (weekly dosing).

In still another aspect, the present invention comprises a transdermal drug delivery device for delivering a pharmaceutically active agent comprising a reservoir comprising a releasably stored dosage of the pharmaceutically active agent, a backing, and a translucent barrier adjacent to the polymeric film backing. The backing is a flexible, translucent polymeric film. The device is characterized in that the moisture vapor transmission rate across the backing and barrier is less than about 2 g/m²/day and the oxygen transmission rate across the backing and barrier is less than about 10 cm³/m²/day. The invention also comprises a method of drug delivery to a mammal comprising providing such a transdermal drug delivery device, placing the device in a delivering relationship to the skin surface of the mammal, and allowing the device to remain in a delivering relationship to the skin for a period of time sufficient to provide a therapeutic effect.

One object of the present invention is to provide a transdermal drug delivery device having a translucent barrier to moisture and oxygen transmission

The oxygen transmission rate (OTR) is a measure of the rate at which oxygen will diffuse through a film under steady-state conditions, and is measured according to ASTM D3895-95. OTR is measured by mounting a film sample as a membrane separating two chambers. One chamber contains oxygen and the other chamber is slowly purged with nitrogen carrier gas. Oxygen diffuses through the film and mixes with the nitrogen carrier gas. The carrier gas is subsequently assayed for oxygen concentration. Oxygen transmission rates reported in the Examples were measured using an Oxtran 1000H (Modern Controls, Inc., MOCON, Minneapolis, Minn.). The oxygen used was HPLC grade. Results were provided as an oxygen transmission rate across the film in units of cm³/m²/day. The diffusion cell area used was 50 cm². The oxygen transmission rate for films used in devices and methods of the present invention is preferably less than about 50 cm³/m²/day, and more preferably less than about 10 cm³/m²/day. Films used in devices and methods of the present invention preferably retain their oxygen transmission barrier properties after being stressed, such as by flexing, folding, or crumpling. The oxygen transmission rate for films used in devices and methods of the present invention preferably does not increase by more than 10-fold, more preferably 5-fold, and most preferably 2-fold, after being subjected to 5 cycles in a Gelbo Flex Tester.

The moisture vapor transmission rate (MVTR) is a measure of the rate at which moisture vapor will diffuse through a film under steady-state conditions, and is measured according to ASTM F 1249-90. MVTR is measured by mounting a film sample as a membrane separating two chambers. One chamber contains moist air and the other chamber is slowly purged with dry carrier gas. Moisture vapor diffuses through the film and mixes with the dry carrier gas. The carrier gas is subsequently assayed for moisture vapor concentration. Moisture vapor transmission rates reported in the Examples were measured using a Permatran-W6 Programmable Water Vapor Permeability Tester (Modem Controls, Inc., MOCON, Minneapolis, Minn.). Results were provided as a moisture vapor transmission rate across the film in units of g/m²/day. Dry nitrogen was used as the carrier gas. HPLC grade water was used in the wet chamber to produce a 100% humidity environment. The diffusion cell area used was 50 cm². The moisture vapor transmission rate for films used in devices and methods of the present invention is preferably less than about 10 g/m²/day, more preferably less than about 5 g/m²/day, and most preferably less than about 2 g/m²/day. Films used in devices and methods of the present invention preferably retain their moisture vapor transmission barrier properties after being stressed, such as by flexing, folding, or crumpling. The moisture vapor transmission rate for films used in devices and methods of the present invention preferably does not increase by more than 10-fold, more preferably 5-fold, and most preferably 2-fold, after being subjected to 5 cycles in a Gelbo Flex Tester.

### EXAMPLES

### Example 1

A 138 nm thick layer of indium tinoxide was deposited by reactive dc magnetron sputtering onto the polyethylene terephthalate side of a 2.0 mil (51 μm) thick laminate film of polyethylene terephthalate (PET) and ethylene vinyl acetate (Scotchpak™ 9732, 3M, St. Paul, Minn.). A 1000 nm thick layer of acrylate was applied to the exposed indium tinoxide surface by flash evaporation, vapor deposition, and crosslinking by electron beam. The resulting film was translucent. Moisture vapor transmission rate and oxygen transmission rate were measured and the results are shown in Table 1. The film was stressed in a Gelbo Flex Tester (Model 5000, United States Testing Corporation) for 0, 1, 2, and 5 cycles. Moisture vapor transmission rate was measured at each interval. Results are shown in Table 2.

### Example 2

A film was prepared following the same general procedure as Example 1, with the exception that the indium tinoxide layer was 35 nm thick. The resulting film was translucent. Moisture vapor transmission rate and oxygen transmission rate were measured and the results are shown in Table 1. The film was stressed in a Gelbo Flex Tester for 0, 1, 2, and 5 cycles. Moisture vapor transmission rate was measured at each interval. Results are shown in Table 2.

### Example 3

A 1000 nm thick layer of acrylate was applied by flash evaporation, vapor deposition, and crosslinking by electron beam onto the polyethylene terephthalate side of a 2.0 mil (51 μm) thick laminate film of polyethylene terephthalate (PET) and ethylene vinyl acetate (Scotchpak™ 9732, 3M, St. Paul, Minn.). A 35 nm thick layer of indium tinoxide was deposited by reactive dc magnetron sputtering onto the exposed surface of the acrylate layer. Another 1000 nm thick layer of acrylate was then applied to the exposed indium tinoxide surface. The resulting film was translucent. Moisture vapor transmission rate and oxygen transmission rate were measured and the results are shown in Table 1.

### Example 4

A film was prepared following the same general procedure as Example 3, with the exception that the acrylate layers were 100 nm thick. The resulting film was translucent. Moisture vapor transmission rate and oxygen transmission rate were measured and the results are shown in Table 1.

### Example 5

A film was prepared following the same general procedure as Example 4, with the exception that the polyethylene terephthalate side of the laminate film was pretreated with a nitrogen plasma at a power level of 500 W prior to application of the acrylate layer. The resulting film was translucent. Moisture vapor transmission rate and oxygen transmission rate were measured and the results are shown in Table 1. The film was stressed in a Gelbo Flex Tester for 0, 1, 2, and 5 cycles. Moisture vapor transmission rate was measured at each interval. Results are shown in Table 2.

### Comparative Example 1

A 2.0 mil (51 μm) thick laminate film of polyethylene terephthalate (PET) and ethylene vinyl acetate (Scotchpak™ 9732, 3M, St. Paul, Minn.) was tested for moisture vapor transmission rate and oxygen transmission rate. Results are shown in Table 1.

TABLE 2

| Ex. No. | MVTR [g/m²/d] | OTR [cm³/m²/day] |
|---|---|---|
| 1 | 0.12 | <0.05 |
| 2 | 0.34 | 1.83 |
| 3 | 0.54 | 1.30 |
| 4 | 0.50 | 2.49 |
| 5 | 0.42 | 2.17 |
| Cl | 21.13 | 106.9 |

| Ex. No. | No. of cycles | MVTR [g/m²/d] |
|---|---|---|
| 1 | 0 | 0.20 |
| | 1 | 0.85 |
| | 2 | 0.58 |
| | 5 | 1.06 |
| 2 | 0 | 0.38 |
| | 1 | 0.79 |
| | 2 | 0.91 |
| | 5 | 0.45 |
| 5 | 0 | 0.89 |
| | 1 | 0.80 |
| | 2 | 1.84 |
| | 5 | 2.40 |

The present invention has been described with reference to several embodiments thereof. The foregoing detailed description and examples have been provided for clarity of understanding only, and no unnecessary limitations are to be understood therefrom. It will be apparent to those skilled in the art that many changes can be made to the described embodiments without departing from the spirit and scope of the invention. Thus, the scope of the invention should not be limited to the exact details of the compositions and structures described herein, but rather by the language of the claims that follow.

We claim:

1. A transdermal drug delivery device for delivering a pharmaceutically active agent comprising:
a) a reservoir comprising a releasably stored dosage of the pharmaceutically active agent; and
b) a substantially continuous, translucent inorganic barrier layer adjacent to at least a portion of the reservoir.

2. A transdermal drug delivery device for delivering a pharmaceutically active agent according to claim 1, further comprising a backing film substrate.

3. A transdermal drug delivery device for delivering a pharmaceutically active agent according to claim 2, wherein the backing film substrate is translucent.

4. A transdermal drug delivery device for delivering a pharmaceutically active agent according to claim 2, wherein the inorganic barrier layer directly adjoins the backing film substrate.

5. A transdermal drug delivery device for delivering a pharmaceutically active agent according to claim 1, further comprising a layer comprising a polymer adjoining the inorganic barrier layer.

6. A transdermal drug delivery device for delivering a pharmaceutically active agent according to claim 5, wherein the polymer is crosslinked.

7. A transdermal drug delivery device for delivering a pharmaceutically active agent according to claim 5, comprising a plurality of inorganic barrier layers.

8. A transdermal drug delivery device for delivering a pharmaceutically active agent according to claim 5, comprising a plurality of layers comprising a polymer.

9. A transdermal drug delivery device for delivering a pharmaceutically active agent according to claim 5, wherein the polymer is a polyacrylate or polymethacrylate.

10. A transdermal drug delivery device for delivering a pharmaceutically active agent according to claim 1, wherein the inorganic barrier layer directly adjoins the reservoir.

11. A transdermal drug delivery device for delivering a pharmaceutically active agent according to claim 1, wherein the inorganic barrier layer is less than about 200 nm thick.

12. A transdermal drug delivery device for delivering a pharmaceutically active agent according to claim 1, wherein the inorganic barrier layer comprises a material selected from the group consisting of indium tinoxide, aluminum oxide, silicon oxide, aluminum-silicon-oxide, aluminum-silicon-nitride, and aluminum-silicon-oxy-nitride.

13. A transdermal drug delivery device for delivering a pharmaceutically active agent according to claim 1, comprising a plurality of inorganic barrier layers.

14. A transdermal drug delivery device for delivering a pharmaceutically active agent according to claim 1, wherein the reservoir comprises a pressure-sensitive adhesive.

15. A transdermal drug delivery device for delivering a pharmaceutically active agent comprising:
a) a reservoir comprising a releasably stored dosage of the pharmaceutically active agent;
b) a flexible, translucent polymeric film backing; and
c) a translucent barrier adjacent to the polymeric film backing,
wherein the device is characterized in that the moisture vapor transmission rate across the backing and barrier is less than about 2 g/m²/day and the oxygen transmission rate across the backing and barrier is less than about 10 cm³/m²/day.

16. A transdermal drug delivery device for delivering a pharmaceutically active agent according to claim 15, wherein the barrier comprises an inorganic barrier layer.

17. A method of drug delivery to a mammal comprising:
a) providing a reservoir comprising a pharmaceutically active agent;
b) providing a substantially continuous, translucent inorganic barrier layer adjacent to at least a portion of one surface of the reservoir:
c) placing the surface of the reservoir opposed to the surface adjacent to the inorganic barrier layer in a delivering relationship to the skin surface of the mammal; and
d) allowing the reservoir to remain in a delivering relationship to the skin for a period of time sufficient to provide a therapeutic effect.

18. A method of drug delivery according to claim 17, wherein the reservoir directly adjoins the skin.

19. A method of drug delivery to a mammal comprising:
a) providing a transdermal drug delivery device according to claim 15;
b) placing the device in a delivering relationship to the skin surface of the mammal; and
c) allowing the device to remain in a delivering relationship to the skin for a period of time sufficient to provide a therapeutic effect.

20. A method of drug delivery according to claim 19, wherein the reservoir directly adjoins the skin.
